(19) United States
(12) Patent Application Publication
(10) Pub. No.: US 2024/0130728 A1
(43) Pub. Date: Apr. 25, 2024

(54) SURGICAL HANDLE ASSEMBLY EXTENDER

(71) Applicant: Lexington Medical, Inc., Bedford, MA (US)

(72) Inventors: David T. Moy, JR., Wellesley, MA (US); Matthew Karee, Cambridge, MA (US); Andrew Marecki, West Boylston, MA (US); Leon Amariglio, Lexington, MA (US)

(73) Assignee: Lexington Medical, Inc., Bedford, MA (US)

(21) Appl. No.: 17/969,378

(22) Filed: Oct. 18, 2022

(51) Int. Cl. A61B 17/068

(52) U.S. Cl. CPC A61B 17/0686; A61B 2017/00367; A61B 2560/0443

(57) ABSTRACT

The present disclosure includes an extender for a surgical stapler. An example extender includes a proximal end configured to removably couple to a surgical handle assembly, a distal end configured to removably coupled to a reloadable cartridge assembly, and an extender drive rod configured to transfer power from the surgical handle assembly to the reloadable cartridge assembly.

### TECHNICAL FIELD

The present disclosure relates generally to a surgical handle assembly, and more particularly, to an extender for a surgical handle assembly.

A surgical handle assembly can be used in a number of surgical devices. One example includes use as a surgical stapler. A surgical stapler is a fastening device used to clamp tissue between opposing jaw structures, separate tissue using a cutting blade, and join tissue using surgical fasteners. In some examples, a surgical stapler can include two elongated members to clamp the tissue. One of the elongated members can include one or more reloadable cartridges and the other elongated member can include an anvil that can be used to form a staple when driven from the reloadable cartridge. A surgical stapler can receive one or more reloadable cartridges. An example of reloadable cartridges can include having rows of staples having a linear length. A row of staples can have a linear length between 30 mm and 60 mm, for example. A staple can be ejected by actuation of a movable handle member that is a part of the surgical handle assembly of the surgical stapler.

For various surgical procedures and based on the size of the patient, surgical handles can come in a variety of lengths. For some procedures and patients, even with the longest length surgical handles available, it may be difficult to position the stapling device in the proper position.

### BRIEF DESCRIPTION OF THE DRAWINGS

FIG. 1 is a schematic diagram of a surgical stapling apparatus including a surgical handle assembly, an extender, and a reloadable cartridge assembly in accordance with a number of embodiments of the present disclosure.

FIG. 2 is an isometric view of an extender in accordance with a number of embodiments of the present disclosure.

FIG. 3 is an exploded view of an extender in accordance with a number of embodiments of the present disclosure.

### DETAILED DESCRIPTION

The present disclosure includes apparatuses for a surgical handle assembly. An example apparatus includes an extender. The extender can be positioned between a surgical handle assembly and a reloadable cartridge assembly when assembled to form a surgical stapler. The extender includes a drive rod and/or an articulation arm. Some extenders include a connector for coupling the drive rod of the extender to the drive mechanism of the surgical handle assembly. Some extenders include a tab extender for coupling the extender articulation arm to the articulation assembly of a reloadable cartridge. In some embodiments, the extender also includes a slider, a block and tab, and/or a blade lock. The slider can aid in disconnecting a reloadable cartridge assembly. The block and tab can indicate when a reloadable cartridge assembly is attached and/or prevent rotation of the drive rod. The blade lock can lock the drive rod when a surgical handle assembly is not attached.

Use of the surgical handle assembly with an extender and a reloadable cartridge assembly can place the reloadable cartridge assembly in either a clamped or an unclamped position in response to a movable handle advancing or retracting the drive system of the surgical stapler. When in the clamped position, further movement of the movable handle will cause staples to be delivered from the reloadable cartridge assembly.

In a number of embodiments, the surgical handle assembly can be constructed in accordance with U.S. Pat. Nos. 10,433,842 and/or 11,103,247, which is incorporated herein by reference and shows, among many embodiments, a mode switch to switch the surgical handle assembly between modes of operation where a switch is configured to provide two or more modes of operation for the movable handle member.

In some embodiments, the blade lock of the extender and/or the blade lock of the reloadable cartridge assembly can be constructed in accordance with U.S. Pat. No. 10,912,560, which is incorporated herein by reference. In some embodiments, the surgical handle assembly can be constructed in accordance with U.S. Pat. No. 10,856,871, which is incorporated herein by reference. In some embodiments, the articulation assembly of the surgical handle assembly can be constructed in accordance with U.S. Pat. No. 11,116,501, which is incorporated herein by reference.

In the following detailed description of the present disclosure, reference is made to the accompanying drawings that form a part hereof, and in which is shown by way of illustration how one or more embodiments of the disclosure may be practiced. These embodiments are described in sufficient detail to enable those of ordinary skill in the al to practice the embodiments of this disclosure, and it is to be understood that other embodiments may be utilized and that process, electrical, and structural changes may be made without departing from the scope of the present disclosure.

It is to be understood that the terminology used herein is for the purpose of describing particular embodiments only and is not intended to be limiting. As used herein, the singular forms “a”, “an”, and “the” can include both singular and plural referents, unless the context clearly dictates otherwise. In addition, “a number of”, “at least one”, and “one or more” (e.g., a number of staples) can refer to one or more staples, whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof means “including, but not limited to”. The terms “coupled” and “coupling” mean to be directly or indirectly connected physically or for access to and/or movement of the movable handle member, as appropriate to the context.

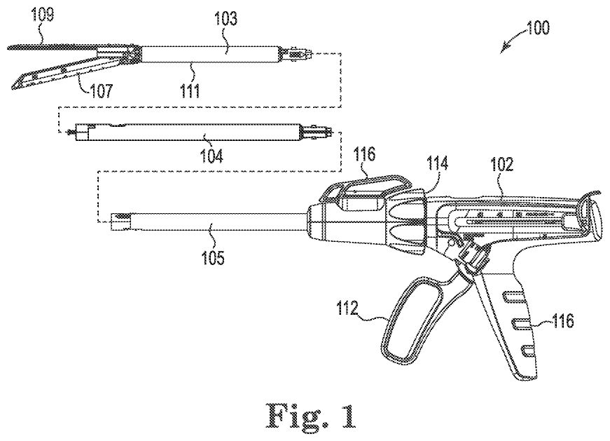

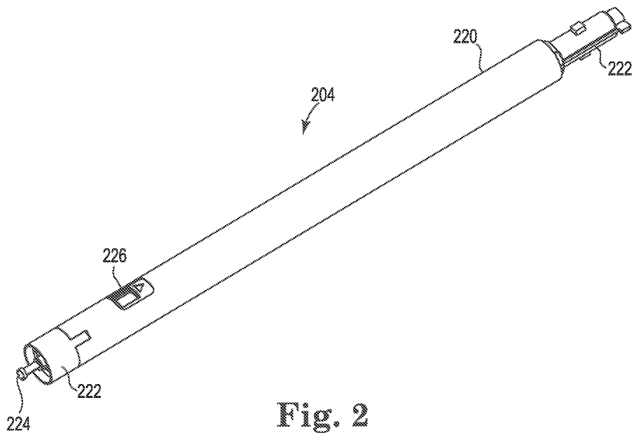

The figures herein follow a numbering convention in which the first digit or digits correspond to the figure number and the remaining digits identify an element or component in the figure. Similar elements or components between different figures may be identified by the use of similar digits. For example, 104 may reference element “04” in FIG. 1, and a similar element may be referenced as 204 in FIG. 2.

As will be appreciated, elements shown in the various embodiments herein can be added, exchanged, and/or eliminated so as to provide a number of additional embodiments of the present disclosure. In addition, the proportion and/or the relative scale of the elements provided in the figures are intended to illustrate certain embodiments of the present disclosure and should not be taken in a limiting sense.

FIG. 1 is a schematic diagram of an apparatus 100 including a surgical handle assembly 102, an extender 104, and a reloadable cartridge assembly 103 in accordance with a number of embodiments of the present disclosure. In some embodiments, the apparatus 100 can be a surgical stapler, for example.

As shown in the example of FIG. 1, the reloadable cartridge assembly 103, e.g., a disposable loading unit, can be releasably secured (e.g., removably coupled) to a distal end of an elongated body of the extender 104. The extender 104 can also be releasably secured to a distal end of an elongated body of the surgical handle assembly 102.

In a number of embodiments, the reloadable cartridge assembly 103 can include a cartridge shaft 111 and a first elongated member 107 and a second elongated member 109 that can be used to clamp tissue. One of the elongated members can house one or more staple cartridges. The other elongated member can have an anvil that can be used to form a staple when driven from the staple cartridge.

As previously mentioned, an apparatus 100 can receive reloadable cartridge assemblies having rows of staples. In some examples, third party reloadable cartridges and/or reloadable cartridge assemblies may be used with the surgical handle assembly 102 and/or extender 104 and embodiments of surgical handle assembly 102 and/or extender 104 may be configured to receive the same.

The reloadable cartridge assembly 103 can be in a clamped position when the first elongated member 107 and the second elongated member 109 are clamping tissue and/or contacting each other. The reloadable cartridge assembly 103 can be in an unclamped position when the first elongated member 107 and the second elongated member 109 are not clamping tissue and/or are not in contact with each other.

The surgical handle assembly 102 coupled to the reloadable cartridge assembly 103 can advance the reloadable cartridge assembly 103 to the clamped position in response to the user actuating a movable handle member 112 of the surgical handle assembly 102 in a proximal direction. The user can also advance the reloadable cartridge assembly 103 to the unclamped position in response to the user actuating the movable handle member 112 in a distal direction.

Along with the movable handle member 112, surgical handle assembly 102 can also include a radial positioner 114, a knob and cam articulation assembly 116, and a handle shaft 105. The extender 104 and reloadable cartridge assembly 103 can be actuated using knob and cam articulation assembly 116 and/or radial positioner 114 to reach a stapling site. The radial positioner 114 can rotate the extender 104 and reloadable cartridge assembly 103. The knob and cam articulation assembly 116 can position the distal end of the reloadable cartridge assembly 103 at a particular angle for stapling. The knob and cam articulation assembly 116 can be configured to actuate rotationally and the first elongated member 107 and the second elongated member 109 can rotate about an axis of a particular plane in response to the knob and cam assembly 116 being actuated rotationally by a user.

In a number of embodiments, the surgical handle assembly 102 coupled to the extender 104 coupled to the reloadable cartridge assembly 103 can deploy a number of staples. The reloadable cartridge assembly 103 can deploy the number of staples in response to the user placing handle assembly 102 in a ‘fire’ mode and moving the movable handle member 112 to the most proximal position. The movable handle member 112 can be in the most proximal position when the movable handle member 112 cannot move further in the proximal direction. Moving the movable handle 112 to the distal most position (or allowing the movable handle 112 to move) followed by moving the movable handle 112 in a proximal direction will cause additional staples to be delivered.

FIG. 2 is an isometric view of an extender 204 in accordance with a number of embodiments of the present disclosure. Extender 204 includes outer tube 220 which covers the extender upper half (not shown) and the extender lower half 222. Extending out the distal end of extender 204 is the distal end of extender drive rod 224. Slider 226 is accessible by a cutout in outer tube 220.

In some embodiments, the length of the extender 204 can be 4, 5, 6, 7, 8, 9, 10, 11, or 12 inches. In other embodiments, the length of the extender 204 can be 7 to 9 inches or 4 to 12 inches, for example.

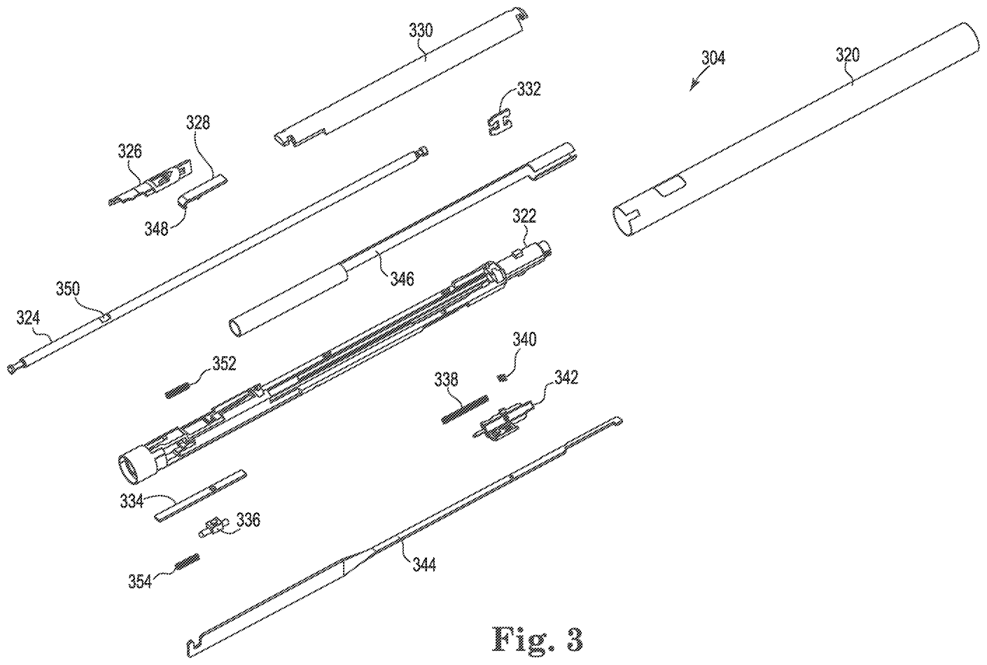

FIG. 3 is an exploded view of extender 304. Extender 304 includes outer tube 320, extender drive rod 324, and, positioned at the proximal end of the extender drive rod 324, connector 332. Connector 332 is configured to connect to the distal end of the drive mechanism (e.g., a drive rod) of a surgical handle assembly (e.g., surgical handle assembly 102 in FIG. 1) and the distal end of extender drive rod 324 is configured to connect to the proximal end of a reloadable cartridge assembly (e.g., reloadable cartridge assembly 103 in FIG. 1). The extender drive rod 324 can transfer force from the surgical handle assembly to the reloadable cartridge assembly. In some embodiments, extender drive rod 324 includes notch 350. In some examples, extender drive rod 324 and connector 332 can be a single piece.

In a number of embodiments, extender 304 also includes extender articulation arm 344. The proximal end of extender articulation arm 344 is configured to connect to the articulation mechanism of the surgical handle assembly and the distal end of the extender articulation arm 344 is configured to connect to the articulation mechanism of the reloadable cartridge assembly. In some embodiments, extender articulation arm 344 is constructed in multiple pieces. The extender articulation arm 344 can transfer articulation movement from the surgical handle assembly to the reloadable cartridge assembly.

In some embodiments, extender 304 can include a blade lock assembly comprising a blade lock 340, a lock slider 342, and a spring 338. In the manufactured configuration, blade lock 340 can be positioned against the distal end of connector 332 to prevent distal movement of the connector 332 and the extender drive rod 324, Extender lower half 322 is configured such that a section is positioned at the proximal end of connector 332 to prevent it from moving proximally. Spring 338 is configured to apply proximal pressure to lock slider 342. When extender 304 is removably coupled to the surgical handle assembly, lock slider 342 will be moved in a distal direction. This distal movement will cause lock slider 342 to interact with blade lock 340 and move the blade lock 340 in a radially outward direction. The movement of blade lock 340 will move it to a position where it no longer interacts with connector 332 and the connector 332 and extender drive rod 324 can be moved in a distal direction. However, this extender 304 is not limited to any particular blade lock assembly and other configurations can be used.

In some embodiments, extender 304 includes a temporary lock mechanism comprising a slider 326, a slider spring 352, and an L-flap 328. L-flap 328 includes a portion 348 that extends in an approximate perpendicular direction from the body of L-flap 328 (it forms an L shape) and a spring (not shown) that biases the L-flap 328 in a radially outward direction. Slider spring 352 is configured to bias slider 326 in a distal direction. In some embodiments, when a reloadable cartridge assembly 103 is removably coupled to extender 304, the proximal end of the reloadable cartridge assembly is first axially pushed against the distal end of the extender 304 and, when fully inserted, rotated. In these embodiments, in order to keep the drive system stable, when the reloadable cartridge assembly first makes contact with the extender 304, slider 326 is pushed proximally over L-flap 328 and portion 348 is forced into notch 350 of the extender drive rod 324. This prevents the extender drive rod 324 from moving axially.

When the user rotates the reloadable cartridge assembly relative to the extender 304, the slider 326 moves distally and releases L-flap 328. In these instances when the reloadable cartridge assembly is removed from the extender 304, the user can move slider 326 proximally over L-flap 328 so that extender drive rod 324 is again prevented from moving axially. This movement assists in the disconnection.

In a number of embodiments, extender 304 includes load detection mechanism which, in some embodiments includes a tab 334, block 336, hypotube 346, and load spring 354. Block 336 is configured with a protrusion that fits within an opening of tab 334 and with a post which supports one side of load spring 354. Hypotube 346, which surrounds drive rod 324 and connector 332, is shown with a cut out section to allow the drive rod 324 and connector 332 to move proximally and distally without any interference. The distal end of hypotube 346 is positioned against tab 334 and the proximal end extends out of extender lower half 322. The load spring 354 provides sufficient force to bias block 336, tab 334, and hypotube 346 in the proximal direction when no reloadable cartridge assembly is attached to the extender 304. If the extender is attached to a surgical handle while not attached to a reloadable cartridge assembly, hypotube 346 will be moved slightly in the distal direction. When a reloadable cartridge assembly is connected to extender 304, block 336, hypotube 346, and tab 334 are moved proximally. When the extender 304 is attached to a surgical handle assembly, this proximal position is relayed to the surgical handle assembly to inform the surgical handle assembly that a reloadable cartridge assembly is attached. In a number of embodiments, tab 334, which has a generally cuboid shape, is positioned against a flat section (not shown) of extender drive rod 324. In this configuration, extender drive rod 324 is prevented from rotating by the tab 334.

In some of the examples described herein, the drive mechanism for the surgical handle assembly is the movable handle (e.g., movable handle 112 in FIG. 1). The embodiments discussed herein are equally applicable to powered handles where an electric motor powers the drive mechanism. In some embodiments, an electric motor is configured to move the drive mechanism while a movable handle 112 is used to clamp and unclamp the elongated members (e.g., elongated members 107 and 109 in FIG. 1).

Some of the examples described herein have a surgical handle assembly with an extender 304 and a reloadable cartridge assembly 103. The embodiments herein are equally applicable to a surgical stapler with a staple cartridge that is loaded into one of the elongated members. The embodiments discussed herein can contain a drive mechanism whose power is derived from axial movement of drive rods including extender drive rod 324. For example, the drive mechanism of the surgical handle assembly can produce axial movement. The extender drive rod 324 can transfer power from the surgical handle assembly to the reloadable cartridge assembly. In some examples, the extender drive rod 324 can transfer power from the surgical handle assembly to the reloadable cartridge assembly by moving axially in response to the axial movement produced by the drive mechanism of the surgical handle assembly.

The embodiments herein are equally applicable to a drive mechanism wherein the power is derived from rotational movement of the extender drive rod 324. For example, the drive mechanism of the surgical handle assembly can produce rotational movement. The extender drive rod 324 can transfer power from the surgical handle assembly to the reloadable cartridge assembly by moving rotationally in response to the rotational movement produced by the drive mechanism of the surgical handle assembly.

In some embodiments, the surgical stapler may include a component configured to move axially to cause the clamping and unclamping of the elongated members and in other embodiments may include a component configured to move rotationally to cause the clamping and unclamping of the elongated members.

When used, the physician/user can assemble the apparatus (e.g., apparatus 100 in FIG. 1) comprising a surgical handle assembly, an extender 304, and a reloadable cartridge assembly prior to use. Once assembled, the distal end of the apparatus can be inserted into a patient via an opening in the body or through a trocar, introducer, or the like. A distal end of the apparatus can be advanced to a target site in the patient. The user may rotate the articulation knob (e.g., articulation knob 116 in FIG. 1) to cause the elongated members to extend away from the longitudinal axis of the apparatus. The user may rotate radial positioner (e.g., radial positioner 114 in FIG. 1) to orient the elongated members properly. When properly positioned, the physician/user can clamp tissue between the elongated members then deliver staples to the tissue at the target site in response to the surgical handle assembly providing axial movement to the extender drive rod 324 and the extender drive rod providing the axial movement to a drive rod of the reloadable cartridge assembly. As the tissue is stapled, a cutting element (not shown) can cut the tissue between the rows of staples. When the usage is complete, the apparatus can be removed from the patient's body.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and processes are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. An extender, comprising:
a proximal end configured to removably couple to a surgical handle assembly;
a distal end configured to removably couple to a reloadable cartridge assembly; and
an extender drive rod configured to transfer power from the surgical handle assembly to the reloadable cartridge assembly.

2. The extender of claim 1, further comprising an extender articulation arm configured to transfer articulation movement from the surgical handle assembly to the reloadable cartridge assembly.

3. The extender of claim 1, wherein the extender drive rod is configured to transfer power from the surgical handle assembly to the reloadable cartridge assembly by moving axially.

4. The extender of claim 1, wherein the extender drive rod is configured to transfer power from the surgical handle assembly to the reloadable cartridge assembly by moving rotationally.

5. The extender of claim 1, further comprising a blade lock assembly.

6. The extender of claim 5, wherein the blade lock assembly comprises a blade lock and a lock slider.

7. The extender of claim 1, further comprising a tab, wherein the tab is configured to prevent the extender drive rod from rotating.

8. The extender of claim 1, further comprising a connector, wherein the connector is positioned on a proximal end of the extender drive rod and is configured to connect to a drive rod of the surgical handle assembly.

9. The extender of claim 1, further comprising a temporary lock mechanism.

10. A surgical stapler, comprising:
a surgical handle assembly;
an extender configured to removably couple to the surgical handle assembly; and
a reloadable cartridge assembly configured to removably couple to the extender.

11. The surgical stapler of claim 10, wherein the extender comprises an extender drive rod for transferring force from the surgical handle assembly to the reloadable cartridge assembly.

12. The surgical stapler of claim 10, further comprising an extender articulation arm configured to transfer articulation movement from the surgical handle assembly to the reloadable cartridge assembly.

13. The surgical stapler of claim 11, wherein a drive mechanism of the surgical handle assembly is configured to produce axial movement and wherein the extender drive rod is configured to move axially in response to the axial movement produced by the drive mechanism of the surgical handle assembly.

14. The surgical stapler of claim 11, wherein a drive mechanism of the surgical handle assembly is configured to produce rotational movement and wherein the extender drive rod is configured to move rotationally in response to the rotational movement produced by the drive mechanism of the surgical handle assembly.

15. The surgical stapler of claim 10, wherein the extender further comprises a blade lock assembly.

16. The surgical stapler of claim 10, wherein the extender further comprises a load detection mechanism.

17. A method of using a surgical stapler, comprising:
providing a surgical handle assembly;
connecting an extender to a distal end of the surgical handle assembly; and
connecting a reloadable cartridge assembly to a distal end of the extender.

18. The method of claim 17, further comprising advancing a distal end of the surgical stapler to a target site in a patient.

19. The method of claim 18, further comprising using a movable handle on the surgical stapler to clamp tissue at the target site and deliver staples to staple tissue at the target site.

20. The method of claim 19, further comprising the surgical handle assembly providing axial movement to an extender drive rod, wherein the extender drive rod provides the axial movement to a drive rod of the reloadable cartridge assembly to deliver the staples.
